(19) United States
(12) Patent Application Publication
(10) Pub. No.: US 2005/0215831 A1
(43) Pub. Date: Sep. 29, 2005

(54) Method for the preparation of polyether alcohols

(75) Inventors: Ralf Zieler, Meuro (DE); Iris Groth, Schwarzheide (DE); Christian Buss, Dresden (DE); Monika Wetterling, Senftenberg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/514,713

(22) PCT Filed: Jun. 18, 2003
(86) PCT No.: PCT/EP03/06405

§ 371 Date: Nov. 23, 2004

(30) Foreign Application Priority Data
Jun. 20, 2002 (DE) 102 27 655.2

(51) Int. Cl. C07C 43/11

(52) U.S. Cl. 568/679

(57) ABSTRACT

The invention relates to a process for the work-up of polyether alcohols which have been prepared by base-catalyzed addition of ethylene oxide and propylene oxide onto H-functional starter substances and have a proportion of ethylene oxide in the polyether chain of greater than 20% by weight, preferably greater than 25% by weight, particularly preferably greater than 35% by weight and in particular greater than 50% by weight, based on the weight of the alkylene oxides used, which comprises the steps a) neutralization of the polyether alcohol with an acid subsequent to the addition reaction of the alkylene oxides, b) addition of at least one absorbent, c) removal of water and volatile compounds from the polyether alcohol, d) removal of the salts formed and the absorbents from the polyether alcohol, wherein phosphoric acid is used as acid in step a) in an amount of from 56 to 98% of the stoichiometric amount, the absorbent is used in an amount of from 0.05 to 5% by weight, based on the polyether alcohol, in step b) and at least one alkali metal hydroxide, alkaline earth metal hydroxide or amine is added to the polyether alcohol in an amount of from 0.001 to 0.5% by weight, based on the weight of the polyetherol, during the course of step c).

The present invention relates to a process for the work-up of polyether alcohols which can be prepared by base-catalyzed addition of alkylene oxides onto H-functional starter substances.

The preparation of polyether alcohols by reaction of H-functional starter substances, in particular alcohols and primary and/or secondary amines, with alkylene oxides is generally known. The reaction of the alkylene oxides with the H-functional starter substances is usually carried out in the presence of catalysts, for example basic or acidic substances or multimetal cyanide catalysts. Potassium hydroxide is usually used as catalyst and is separated off from the polyether alcohol after the synthesis by purification operations such as neutralization, distillation and/or filtration. Only these pure polyether polyols are used for the reaction with the diisocyanates and/or polyisocyanates.

The work-up can be carried out, for example, by addition of solid compounds having acid centers, e.g. ion exchangers or aluminosilicates, to the polyether alcohol and their subsequent removal, for example by means of filtration or by means of centrifugation. However, owing to the low effectiveness of these compounds, a large amount of such compounds has to be added and subsequently separated off again.

A further possible way of carrying out the work-up is neutralization of the basic catalysts by means of an acid and removal of the salts formed by means of a suitable separation process, for example by means of filtration or by means of centrifugation.

A series of processes for the work-up of polyether alcohols using acids are known from the prior art.

When acids are used as neutralizing agents, the setting of the neutral point presents a particular problem.

Thus, U.S. Pat. No. 3,016,404 describes the use of hydrochloric acid as neutralizing agent, with the hydrochloric acid being added in excess and the excess being stripped out as hydrogen chloride. However, due to the corrosive nature of hydrochloric acid, its use places severe demands on the apparatus used. In addition, this process results in the liberation of gaseous hydrogen chloride, which represents a potential safety hazard.

In the case of polyfunctional acids such as sulfuric acid or phosphoric acid, the setting of the neutral point is significantly more difficult because of the multiple dissociation of the acids.

To solve this problem, DE-A-32 29 216 proposes using phosphoric acid in combination with an adsorbent. Adsorbents used are, in particular, natural or synthetic silicates. However, this process is unreliable in terms of the setting of the neutral point. In addition, particularly in the case of polyether alcohols having a high ethylene oxide content, formation of unfilterable crystal turbidity and leaching of undesirable metal ions from the silicates can occur.

DE-A-14 95 719 describes the purification of polyether alcohols by means of phosphoric acid, with removal of the water and the salts being carried out under a precisely defined pressure and temperature regime. Organic solvents are preferably used in this process. However, this process is cumbersome and in the case of polyether alcohols having a high proportion of ethylene oxide in the chain leads to turbidity in the polyether alcohol.

WO 99/47582 describes a process for preparing low-odor polyether alcohols. Here, the neutralized or unneutralized polyether alcohol is brought into contact with an acid and water is subsequently added under hydrolysis conditions. In this process, it is possible for the acid to be added in excess. The excess acid can subsequently be neutralized by means of alkaline compounds, in particular alkali metal hydroxides. The salts formed are separated off by means of filtration. In this process, too, crystal turbidity and, associated therewith, difficulties in removing the salts from the polyether alcohol occur, especially in the purification of polyether alcohols having a high ethylene oxide content in the chain. The excess of acid necessary in the hydrolysis of the odor-imparting substances present in the polyether alcohol and, resulting therefrom, the further addition of alkaline compounds to reneutralize the excess acid makes it very difficult to set the alkalinity, i.e. the content of potassium and sodium ions in the polyether alcohol.

Polyether alcohols having a high ethylene oxide content, in particular an ethylene oxide content of greater than 20% by weight, preferably greater than 25% by weight, particularly preferably greater than 35% by weight and in particular greater than 50% by weight, based on the weight of the polyether alcohol, are preferably used for producing flexible polyurethane foams, in particular cold-cure molded foams. Here, the ethylene oxide can be distributed over the entire polyether chain. In the case of polyether alcohols which are intended for the production of cold-cure molded foams, it is preferred that at least part of the ethylene oxide is added on at the end of the chain.

Polyether alcohols having a high proportion of ethylene oxide in the chain are very hydrophilic and therefore make the customary work-up difficult. The unfavorable solubility of the salts formed after the neutralization in the polyether alcohol causes a particular problem in the purification, and an undesirably high content of alkali metal ions in the finished polyether alcohol can result.

It is an object of the present invention to provide a process for the work-up of polyether alcohols, in particular those which have been prepared by base-catalyzed addition of ethylene oxide and propylene oxide onto H-functional starter substances and have a proportion of ethylene oxide in the polyether chain of at least 20% by weight, based on the weight of the alkylene oxides used, without turbidity occurring and an undesirably high proportion of the fine salt crystals being obtained and with virtually complete removal of the alkali metal ions from the polyether alcohol being achieved.

In the neutralization, the amount of acid, for example phosphoric acid, required for neutralization of the alkali metal hydroxide is based on the formation of primary salts, i.e. those containing one metal ion. The objective is to form the major part of the salts with this amount of acid, since these salts can be separated most readily from the polyether alcohol, in particular in the case of phosphates. The formation of readily filterable salts is usually ensured only in a particular pH range, in the case of phosphoric acid from 6 to 7. Setting such a pH usually proves to be difficult in practice and is tremendously time consuming.

We have found that the object of the present invention is achieved by using phosphoric acid as acid in the purification of the polyether alcohol and carrying out the neutralization in the presence of absorbents such as cellulose, active earths, ion exchangers and/or bentonites and adding a basic compound to the polyether alcohol during the final removal of water and volatile substances.

The present invention accordingly provides a process for the work-up of polyether alcohols which have been prepared by base-catalyzed addition of ethylene oxide and propylene oxide onto H-functional starter substances and have a proportion of ethylene oxide in the polyether chain of greater than 20% by weight, preferably greater than 25% by weight, particularly preferably greater than 35% by weight and in particular greater than 50% by weight, based on the weight of the alkylene oxides used, which comprises the steps
a) neutralization of the polyether alcohol with an acid subsequent to the addition reaction of the alkylene oxides,
b) addition of at least one absorbent,
c) removal of water and volatile compounds from the polyether alcohol,
d) removal of the salts formed and the absorbents from the polyether alcohol,
   wherein phosphoric acid, preferably phosphoric acid having a concentration in the range from 80 to 85%, is used as acid in step a) in an amount of from 56 to 98% of the stoichiometric amount, the absorbent is used in an amount of from 0.05 to 5% by weight, based on the polyether alcohol, in step b) and at least one alkali metal hydroxide, alkaline earth metal hydroxide or amine is added to the polyether alcohol in an amount of from 0.001 to 0.5% of the stoichiometric amount during the course of step c).

The absorbent in step b) is preferably selected from the group consisting of cellulose, earths and active earths, ion exchangers, bentonites and silicates.

The steps a) and b) can be carried out simultaneously. However, it is also possible to commence the work-up of the present invention at step a) or step b). For the purposes of the present invention the term “stoichiometric amount” means that one mol of phosphoric acid is used per mole of alkali metal hydroxide.

The polyether alcohol is preferably admixed with water prior to the addition of the phosphoric acid. The amount of water added is, in particular, in a range from 1 to 20% by weight, based on the weight of the polyether alcohol.

The neutralization in step a) is preferably carried out at from 60 to 130° C. During the neutralization, the reactor should be blanketed with inert gas, in particular nitrogen. The neutralization generally occurs over a period of from 1 to 4 hours.

The subsequent removal of water, generally down to a water content of <0.1%, and volatile constituents from the polyether alcohol is preferably carried out by distillation, in particular under reduced pressure, or by stripping with an inert gas, preferably nitrogen. This treatment is preferably carried out at from 100 to 130° C.

The addition of alkali metal hydroxide, alkaline earth metal hydroxide or amine in step c) is preferably carried out at a water content in the polyether in the range from 0.1 to 0.5% by weight, in particular from 0.2 to 5% by weight. The conditions for stripping can in principle remain constant or be altered before, during or after the addition of the alkali metal hydroxide.

The cellulose used as absorbent preferably has a fiber diameter in the range from 3 nm to 1 μm. The cellulose is usually made up of loose white fibers or short white fibers having a dry content of from 90 to 95% and is preferably highly pure and extract-free and has a bulk density of from 20 to 200 g/l. The salts formed are separated off in step d) by means of, in particular, filtration. A filter aid can be used here if desired. Cellulose in particular can be used as filter aid. In principle, the same cellulose as has been used as absorbent can also be used as filter aid.

The process of the present invention can be employed to prepare, in particular, polyether alcohols for producing flexible polyurethane foams and rigid polyurethane foams. As stated above, the polyether alcohols which have been worked up by the process of the present invention and have a high ethylene oxide content are particularly useful for producing flexible polyurethane foams.

In the preparation of polyether alcohols which are used for producing flexible polyurethane foams, the starter substances used are usually alcohols having 2 or 3 hydroxyl groups. Preferred starter substances are glycerol, trimethylolpropane, ethylene glycol, diethylene glycol, propylene glycol, dipropylene glycol and any mixtures of at least two of the alcohols mentioned. As alkylene oxides, ethylene oxide and propylene oxide are used together. The alkylene oxides can be added on individually in succession as blocks or randomly as mixtures with one another. For particular applications, for example for the production of cold-cure molded foams, an ethylene oxide block can be added on at the end of the polyether chain. The polyether alcohols which are used for producing flexible polyurethane foams usually have a molecular weight Mn in the range from 1 000 to 10 000 g/mol, in particular from 1 000 to 7 000 g/mol.

In the case of polyether alcohols which are used for producing rigid polyurethane foams, use is usually made of starter substances having at least 4 active hydrogen atoms, preferably at least 4-hydric alcohols and/or amines having at least 4 reactive hydrogen atoms. It is possible to use either aliphatic or aromatic amines. Preference is given to aromatic amines.

Examples of at least 4-hydric alcohols are sugar alcohols such as glucose, sorbitol, sucrose and mannitol. Since these compounds are usually solid, they are usually admixed with liquid compounds such as water, glycerol and/or ethylene glycol for the reaction with the alkylene oxides. In the process of the present invention, it is in principle also possible to use mixtures of the abovementioned solid compounds and the amines used according to the present invention as starter substance.

As aromatic amines, use is usually made of toluenediamine, diphenylmethanediamine and mixtures of diphenylmethanediamine and polyphenylene-polymethylene polyamines. As aliphatic amines, use is usually made of ethylenediamine, diethylenetriamine, dimethylpropylamine or their higher homologues.

To commence the reaction, the basic catalyst, which is preferably used in an amount of from 0.05 to 0.5% by weight, more preferably from 0.1 to 0.3% by weight, based on the weight of the polyether alcohol, is mixed with the starter substance and the resulting mixture is, if appropriate, subjected to distillation to remove water and volatile constituents. The alkylene oxides are then introduced.

The reaction of the starter substance with the alkylene oxides is carried out at the customary pressures in the range from 0.1 to 1.0 MPa and the customary temperatures in the range from 80 to 140° C. The introduction of alkylene oxides is usually followed by an after-reaction phase to allow the alkylene oxides to react completely. In an advantageous embodiment of the process of the present invention, additional catalyst is added to the reaction mixture at the beginning of the after-reaction phase, preferably immediately after introduction of the alkylene oxides has been concluded.

After the addition reaction with the alkylene oxides, the polyether alcohols are freed of catalyst by the process of the present invention.

The customary stabilizers are then usually added to the polyether alcohol in the amounts customary for this purpose.

The polyether alcohols which have been worked up by the process of the present invention are usually used for producing polyurethanes. For this purpose, they are reacted with polyisocyanates, preferably in the presence of catalysts and, if polyurethane foams are being produced, of blowing agents and, if desired, customary and known auxiliaries and/or additives.

The process of the present invention for working up the polyether alcohols surprisingly makes it possible to neutralize polyether alcohols having a high ethylene oxide content in the polyether chain without turbidity phenomena occurring and to purify them without problems in adjusting the pH. Furthermore, no problems with very fine salt particles which cannot be filtered occur.

The polyether alcohols which have been worked up by the process of the present invention have low residual salt contents and a low acid number. Thus, a potassium value of not more than 10 mg of K/kg and an acid number of not more than 0.03 mg of KOH/g can be achieved.

The preferred use of the polyether alcohols which have been worked up by the process of the present invention is in the production of polyurethanes. For this purpose, the polyether alcohols are reacted with polyisocyanates, if appropriate in the presence of catalysts, blowing agents and customary auxiliaries and additives. The polyether alcohols having high ethylene oxide contents are preferably used for producing flexible polyurethane foams, in particular molded foams and most preferably cold-cure foams. They are particularly useful as cell openers in such foams.

The process is illustrated by the following examples:

### COMPARATIVE EXAMPLE 1

1 kg of an alkaline crude polyether alcohol based on glycerol and a mixture of propylene oxide and ethylene oxide and having an ethylene oxide content of 35% by weight and an ethylene end block amounting to 25% by weight, an OH number of 45 mg of KOH/g and an alkalinity of 0.3% by weight of KOH, based on the crude polyether, was admixed with 80 g of water and 5.95 g of 80% strength phosphoric acid and stirred at 90° C. under a blanket of nitrogen for 2 hours, with crystal turbidity occurring. Even after the subsequent vacuum distillation at 120° C. and filtration, the turbidity remained. The finished product had a water content of 0.08% by weight, a pH of 7.5 and a potassium value of 80 mg of K/kg.

### COMPARATIVE EXAMPLE 2

1 kg of an alkaline crude polyether alcohol based on glycerol and a mixture of propylene oxide and ethylene oxide and having an ethylene oxide content of 60% by weight and an ethylene end block amounting to 22% by weight, an OH number of 38 mg of KOH/g and an alkalinity of 0.15% by weight of KOH, based on the crude polyether, was admixed with 40 g of water and 1.96 g of 80% strength phosphoric acid and stirred at 90° C. under a blanket of nitrogen for 2 hours, with crystal turbidity occurring. Even after the subsequent vacuum distillation at 120° C. and filtration, the turbidity remained. The finished product had a water content of 0.05% by weight, a pH of 8.5 and a potassium value of 200 mg of K/kg.

### COMPARATIVE EXAMPLE 3

1 kg of an alkaline crude polyether alcohol based on glycerol and a mixture of propylene oxide and ethylene oxide and having an ethylene oxide content of 55% by weight and an ethylene end block amounting to 15% by weight, an OH number of 45 mg of KOH/g and an alkalinity of 0.5% by weight of KOH, based on the crude polyether, was admixed with 90 g of water and 10.8 g of 80% strength phosphoric acid and stirred at 90° C. under a blanket of nitrogen for 2 hours, with crystal turbidity occurring. After neutralization, 10 g of colloidal magnesium silicate Ambosol® from Ambozel were added. Even after the subsequent vacuum distillation at 120° C. and filtration, the turbidity remained. The finished product had a water content of 0.08% by weight, a pH of 7.5 and a potassium value of 22 mg of K/kg.

### EXAMPLE 1

1 kg of an alkaline crude polyether alcohol based on glycerol and a mixture of propylene oxide and ethylene oxide and having an ethylene oxide content of 35% by weight and an ethylene end block amounting to 25% by weight, an OH number of 45 mg of KOH/g and an alkalinity of 0.3% by weight of KOH, based on the crude polyether, was admixed with 80 g of water and 1 g of Arbocel made up of short white fibers and having a bulk density of 160 g/l and a residual moisture content of 5% and then 5.95 g of 80% strength phosphoric acid and stirred at 90° C. under a blanket of nitrogen for 0.2 hours. Slight crystal turbidity occurred here.

After removal of water to a residual content of 0.1% by means of vacuum distillation under the conditions described in comparative example 1, 0.04 g of 48% strength KOH was added. After addition of KOH and continuation of the vacuum distillation to a water content of 0.08% by weight, the crystal turbidity had disappeared. After subsequent filtration, the finished product had a water content of 0.08% by weight, a pH of 7.5 and a potassium value of 8 mg of K/kg.

### EXAMPLE 2

1 kg of an alkaline crude polyether alcohol based on glycerol and a mixture of propylene oxide and ethylene oxide and having an ethylene oxide content of 60% by weight and an ethylene end block amounting to 22% by weight, an OH number of 38 mg of KOH/g and an alkalinity of 0.15% by weight of KOH, based on the crude polyether, was admixed with 40 g of water and 0.8 g of “Makrosorb MP 5” absorbent from Crosfield B.V. having a potassium hydroxide absorption potential of 180 mg of KOH/g and subsequently 1.96 g of 80% strength phosphoric acid and stirred at 90° C. under a blanket of nitrogen for 2 hours.

Slight crystal turbidity occurred here.

After removal of water to a residual content of 0.1% by means of vacuum distillation under the conditions described in comparative example 2, 0.02 g of 48% strength KOH was added. After addition of KOH and continuation of the vacuum distillation to a water content of 0.05% by weight, the crystal turbidity had disappeared. After subsequent filtration, the finished product had a water content of 0.05% by weight, a pH of 6.5 and a potassium value of 3 mg of K/kg.

### EXAMPLE 3

1 kg of an alkaline crude polyether alcohol based on glycerol and a mixture of propylene oxide and ethylene oxide and having an ethylene oxide content of 55% by weight and an ethylene end block amounting to 15% by weight, an OH number of 45 mg of KOH/g and an alkalinity of 0.5% by weight of KOH, based on the crude polyether, was admixed with 90 g of water and 10.8 g of 80% strength phosphoric acid and stirred at 90° C. under a blanket of nitrogen for 2 hours, with crystal turbidity occurring. After neutralization, 10 g of colloidal magnesium silicate Ambosol® from Ambozel were added. Even after the subsequent vacuum distillation at 120° C. and filtration, the turbidity remained.

After removal of water to a residual content of 0.3% by means of vacuum distillation under the conditions described in comparative example 3, 100 g of 48% strength KOH were added. After addition of KOH and continuation of the vacuum distillation to a water content of 0.05% by weight, the crystal turbidity had disappeared. After subsequent filtration, the finished product had a water content of 0.05% by weight, a pH of 7.0 and a potassium value of 7 mg of K/kg.

1. A process for the work-up of polyether alcohols which have been prepared by base-catalyzed addition of ethylene oxide and propylene oxide onto H-functional starter substances and have a proportion of ethylene oxide in the polyether chain of greater than 20% by weight, based on the weight of the alkylene oxides used, which comprises the steps
a) neutralization of the polyether alcohol with an acid subsequent to the addition reaction of the alkylene oxides,
b) addition of at least one absorbent,
c) removal of water and volatile compounds from the polyether alcohol,
d) removal of the salts formed and the absorbents from the polyether alcohol,
wherein phosphoric acid is used as acid in step a) in an amount of from 56 to 98% of the stoichiometric amount, the absorbent is used in an amount of from 0.05 to 5% by weight, based on the polyether alcohol, in step b) and at least one alkali metal hydroxide, alkaline earth metal hydroxide or amine is added to the polyether alcohol in an amount of from 0.001 to 0.5% by weight, based on the weight of the polyetherol, during the course of step c) and the process commences with steps a) and/or b).

2. A process as claimed in claim 1, wherein the absorbent in step b) is selected from the group consisting of cellulose, ion exchangers, bentonites and silicates.

3. A process as claimed in claim 1, wherein the addition of the alkali metal hydroxide, alkaline earth metal hydroxide and/or amine in step c) is preferably carried out at a water content in the polyether alcohol in the range from 0.1 to 0.5% by weight.

4. A process as claimed in claim 1, wherein the salts formed are separated off in step d) by means of filtration.

5. A process as claimed in claim 4, wherein the salts formed are separated off in step d) in the presence of a filter aid.
